(19) United States
(12) Patent Application Publication
(10) Pub. No.: US 2006/0293630 A1
(43) Pub. Date: Dec. 28, 2006

(54) Fluid containment apparatus for surgery and method of use

(75) Inventors: Ronald R. Manna, Valley Stream, NY (US); Dan Voic, Clifton, NJ (US); Theodore A. D. Novak, Northport, NY (US); Scott Isola, Deer Park, NY (US); Christopher Bush, Commack, NY (US); Steven Tobias, East Meadow, NY (US)

Correspondence Address: COLEMAN SUDOL SAPONE, P.C., 714 COLORADO AVENUE, BRIDGE PORT, CT, 06605-1601, US

(73) Assignee: Misonix Incorporated, Farmingdale, NY (US)

(21) Appl. No.: 11/158,883

(22) Filed: Jun. 22, 2005

(51) Int. Cl. A61M 1/00

(52) U.S. Cl. 604/327

(57) ABSTRACT

A liquid containment system in accordance with the present invention includes a flexible plastic or elastomeric bag with several apertures. Surrounding each aperture is a port member such as a silastic ring. The port member functions at least reduce the escape of airborne particules from the bag while permitting the insertion of a surgical instrument into the bag. The port member may be made at least partially of a silicone based material to provide resistance to damage by ultrasonic tools.

### BACKGROUND OF THE INVENTION

This invention relates to a device and a system for containment of both airborne particles and waste liquid generated during surgical procedures. The device and system of this invention are particularly appropriate for the containment of atomized particles and waste liquid generated during ultrasonic surgical procedures. The invention also pertains to associated methods of use.

Over the past 30 years, several ultrasonic tools have been invented which can be used to ablate or cut tissue in surgery. Such devices are disclosed by Wuchinich et al. in U.S. Pat. No. 4,223,676 and Idemoto et al. in U.S. Pat. No. 5,188,102.

In practice, these surgical devices include a blunt tip hollow probe that vibrates at frequencies between 20 kc and 100 kc, with amplitudes up to 300 microns or more. Such devices ablate tissue by either producing cavitation bubbles which implode and disrupt cells, tissue compression and relaxation stresses (sometimes called the jackhammer effect), or by other forces such as micro streaming of bubbles in the tissue matrix. The effect is that the tissue becomes liquefied and separated. The tissue then becomes emulsified with the irrigant solution. The resulting emulsion is then aspirated from the site. Bulk excision of tissue such as a tumor is possible by applying the energy around and under the tumor to separate it from the surrounding structure. The surgeon can then lift the tissue out using common tools such as forceps.

The probe or tube is excited by a transducer of either the piezoelectric or magnetostrictive type that transforms an alternating electrical signal within the frequencies indicated into a longitudinal or transverse vibration. When the probe is attached to the transducer, the two become a single element with series and parallel resonances. The designer will try to tailor the mechanical and electrical characteristics of these elements to provide the proper frequency of operation. Most of the time, the elements will have a long axis that is straight and has the tip truncated in a plane perpendicular to the long axis. This is done for simplicity and economic considerations. In almost all applications, whether medical or industrial, such an embodiment is practical and useful.

However, when the devices are used in open field surgery, particularly when the operative sight is on the skin or slightly below, a side effect of the ultrasonic probe interaction with fluids becomes apparent. This is the ability of the ultrasonic vibrating tool to break up fluid and aerosol them such that the particles of fluid may be projected into the operating room atmosphere and either inhaled or otherwise contaminate people or surfaces.

The ability of ultrasonic probes to atomize fluid is well known. Many patents have been issued for just such hardware, such as U.S. Pat. Nos. 5,516,043, 4,153,201, 4,337,896, 4,541,564, among many others. These devices have application in industry and medicine where it is desired to create fine particles of fluid and transport them to an airstream. In operation, a fluid is introduced to the distal end of the vibrating probe. The frequency of vibration may be from 20 to 200 kHz or higher. The vibrating tip breaks the surface tension of the fluid and projects small amounts of fluid off the surface. The natural phenomenon of fluid to coalesce to a sphere in space creates small particles, which by their low mass and aerodynamic nature are easily suspended in air. Depending upon variables such as frequency of vibration, amplitude and liquid properties, ultrasonic atomizers can produce aerosols with mean diameters from hundreds of microns to below 1 micron in size.

In the operating room, this atomization has caused concern since the fluids being atomized can contain blood, virus, bacteria or other objectionable constituents. When the probe is in contact with the tissue, no atomization of the liquid occurs from the distal portion of the probe to the work surface. However, the liquid and tissue is also present on the rear faces of asymmetrical probes and the sides of the probes as well. These surfaces project the spray away from the work surface and into the air. In addition, the tissue being ablated by the direct contact with the probe may be aerosolized as well. This phenomenon of aerosoling contaminants is not limited to ultrasonic aspirators alone. Medical lasers can produce smoke when ablating tissue that contains viruses as well. It is well documented that the virus that causes vaginal warts may be present in laser smoke. When the smoke contacts operating room personnel, warts will appear on lips and other mucous membranes. Several inventions have been developed to combat this smoke problem. One such device is the smoke extractor unit. This is a vacuum pump, chemical filter and pickup hose assembly. In practice, the pickup funnel is placed near the operative sight. The air surrounding the site is sucked into the extractor funnel, similar to a vacuum cleaner effect. The smoke and objectionable elements are removed with the chemical filter. The air is then exhausted back into the room.

Although these devices work reasonably well with laser smoke, they do not provide solutions to all of the issues involved with ultrasonic surgery. For one, the aerosols emitted by the ultrasonic probe are liquid in nature and will contaminate the chemical filter. The liquid will drip out of the filter, causing contamination that must be disinfected and cleansed. Another problem encountered with ultrasonic aspirators not found in lasers is the liquid irrigation fluid waste created.

In all ultrasonic aspirators, liquid is added to the site to increase the ultrasonic action and to flush the field of debris. If a separate aspiration wand is not used, especially on burn wounds or bedsores, the liquid will run off of the patient and onto the floor. Even in cases where a separate wand is used, the amount of fluid is excessive such that not all of the fluid may be aspirated. This causes contamination that must be cleaned.

It is therefore desired to find a means to reduce the escape of aerosols into the general operating room atmosphere and to contain the liquid drain off while not impeding the line of sight of the surgeon nor impeding convenient access to the wound.

### OBJECTS OF THE INVENTION

An object of the present invention is to provide an improved ultrasonic surgical method for use in debridement of wounds or ablation of tissue in open field procedures.

Another object of the present invention is to provide a means to contain the aerosols generated by ultrasonic surgical aspirators when used to debride wounds or ablate tissue in open field procedures.

Another relatively specific object of the present invention is to provide a means to contain and collect contaminated liquid effluent for at least some kinds of ultrasonic ablation or debridement procedures.

It is a further object of the present invention to provide such a means for aerosol and liquid effluent containment without restricting access to wound site nor impeding the surgeon's line of sight.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

A liquid containment system in accordance with the present invention includes a flexible plastic or elastomeric bag with several apertures. Surrounding each aperture is a port member such as a silastic ring. The port member is designed to at least reduce the escape of airborne particules from the bag while permitting the insertion of a surgical instrument into the bag. Where the surgical instrument is an ultrasonic tool, it is preferred that the port member is made at least partially of a silicone based material due to the inherent resistance of such materials to damage by ultrasonic tools. However, the invention is not restricted to these materials, since other materials may provide the same benefits.

The bag, which ideally should be made from a clear or highly translucent material, has an opening which incorporates either an elastic band mounted circumferentially around the opening or a draw string device, either of which can be used to tighten the opening around a limb or torso of the of the patient, and thus inhibit the egress of liquid and airborne particles from the bag via the opening.

During the preparation of the patient for surgery, the bag is removed from its packaging. The bag may be sterile or unsterile, depending upon the type of surgery to be performed. The open end of the bag is then slid up over the limb to be operated on. In the case of torsos, posteriors or upper thighs, the bag may be shaped in the rough approximation of a body suit.

The bag or suit must be oversized such that a good deal of play is available in the skin of the bag. This play will allow the opening(s) present in the bag to be positioned over the wound or surgical site. The bag may be adjusted around the patient to facilitate this placement before the drawstring is tightened around the patient to fix the bag in place. In the case where an elastic band is used, the elastic band may be stretched opened and slid around the body to accomplish the same thing.

The silastic rings may be of different shapes and sizes. They may incorporate flaps or fingers to trap more aerosol particles. They may be raised and have drain ports incorporated. They may have screw tops to seal the openings so that they are leak tight, allowing more than one opening to be located on the bag.

In any case, one or more drain ports with liquid tight seals will be incorporated in the bag. When the bag is positioned on the patient, the drain port will be positioned at the lowest possible point. This will allow an aspiration tube to be placed on the drain port. When connected to a standard aspiration system with collection bottle attached, the liquid will be drained out of the bag to the collection bottle.

If the surgical opening is fairly large, the pressure differential from the bag interior to the atmosphere will be low enough not to cause the bag to collapse around the patient. Even with a small amount of collapse, the bag should be sized such that air paths are present to allow full flow through the opening. In cases were it is desired to keep the surgical opening very small, vent flaps may be added to the bag to allow airflow though it to prevent collapse.

Alternatively, the bag may be supported by rigid members to provide structural support, such as wires or ribs. Such supports may be taped, glued, molded into or otherwise integrated into the assembly. The supports may be used to shape the bag to aid the surgical procedure.

The flow through the silastic opening causes a vacuum cleaner effect in that the aerosols will be captured and brought back into the bag. Once the atomized fluids contact the wall of the bag, they will coalesce and be removed from the drain port. Even if suction is not used, the raised section of the bag has been shown to reduce the escape of atomized fluids substantially.

After the procedure is through, the bag is removed from the patient and discarded along with all other contaminated garments and articles.

In testing, the bag system has proven effective and advantageous for the reasons stated.

### BRIEF DESCRIPTION OF THE DRAWINGS

FIG. 1 is a schematic perspective view of a human appendage with an open wound.

FIG. 2 is a schematic perspective view similar to FIG. 1, showing a surgical fluids containment bag in accordance with the present invention in place about the appendage.

FIG. 3 is a schematic perspective view of a portion of the containment bag of FIG. 2, showing a plurality of silastic port members provided at respective access apertures or openings in the bag.

FIG. 4 is a view similar to FIG. 2,, showing the bag with multiple silastic port members at openings around the bag.

FIG. 5 is a schematic perspective view of a surgical fluids containment bag for a lower torso.

FIG. 6 is a schematic perspective view similar to FIG. 4, showing a fluid collection system connected to a fluid collection valve on the surgical fluids containment bag.

FIG. 7 is a schematic perspective view similar to FIG. 2, showing the fluids containment bag with positive ventilation.

FIG. 8 is a schematic perspective view of a surgical fluids containment bag for localized wound access.

### DETAILED DESCRIPTION

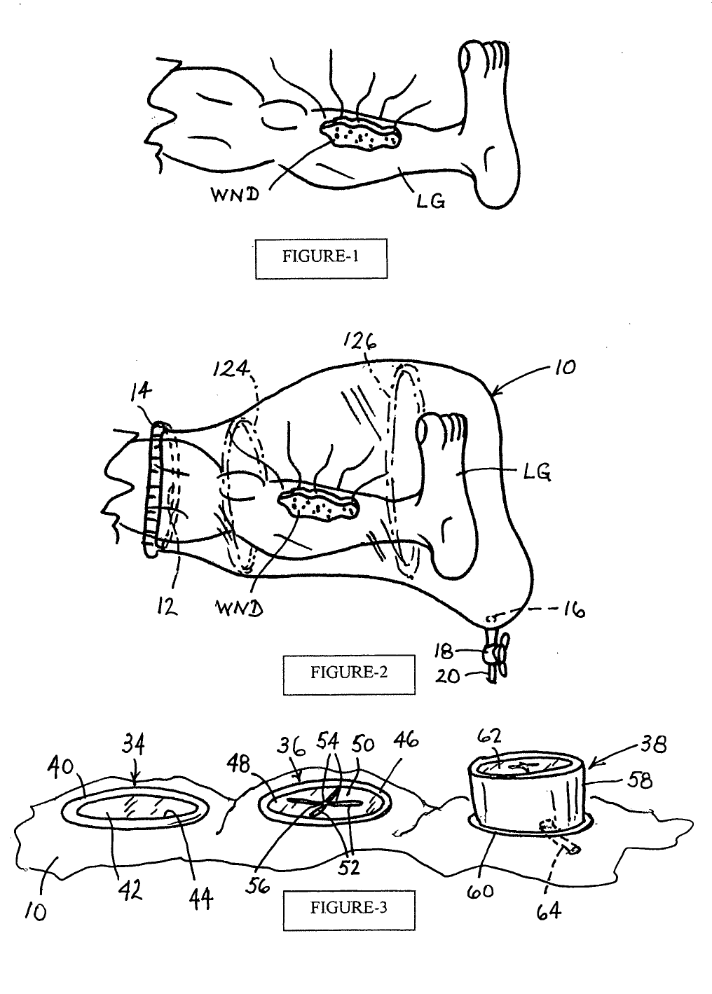

As illustrated in FIG. 1, an appendage LG of a patient may have a wound or trauma WND which requires a surgical procedure such as ultrasonic ablation or wound debridement. In order to capture airborne particles and liquid effluent generated by the ultrasonic ablation or debridement procedure, a fluids containment bag 10 is placed about the appendage LG. Bag 10 has a mouth or opening 12 provided around a rim (not separately enumerated) with an elastic band or drawstring 14 for closing the mouth or opening 12 about the patient to effectuate a preferably fluid-tight engagement with the patient.

Fluids containment bag 10 is provided, for instance, at an end opposite opening 12, with an aperture or opening 16 for the drainage of liquid from the bag. Such liquid may include blood, as well as saline or other liquid that is applied to the surgical site, for example, for cooling and cleaning purposes. Bag 10 is provided at opening 16 with a valve 18 and a connector 20.

Bag 10 is made at least partially of optically transmitting material. Preferably the entire bag is made of substantially transparent material for facilitating visual observation of appendage LG and surgical procedures effectuated on wound WND. Alternatively, bag 10 may be partially opaque and provided with one or more windows of transparent or translucent plastic film material.

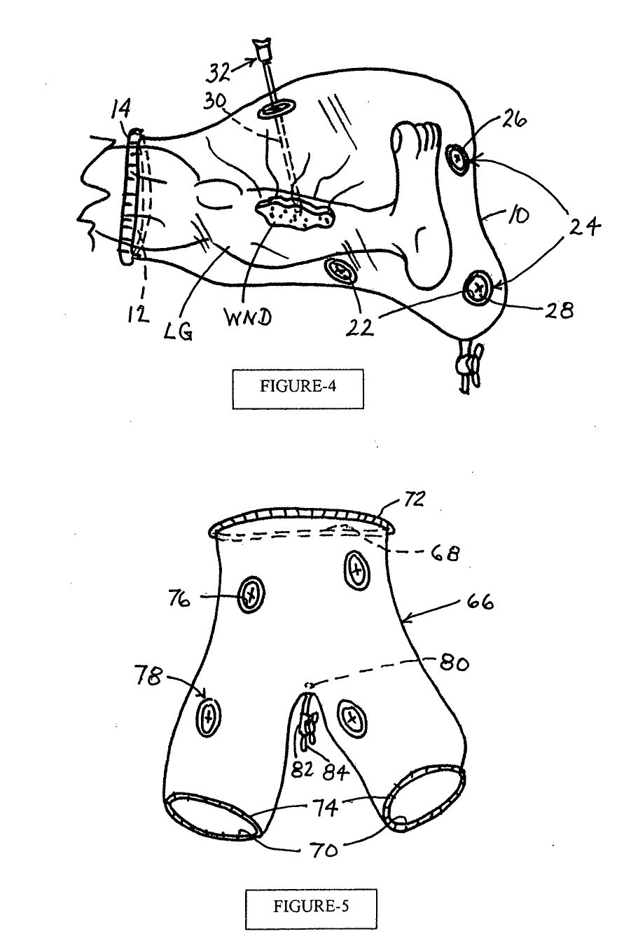

As depicted in FIG. 4, bag 10 is provided at mutually spaced locations with a plurality of openings 22 defined by respective port members 24 exemplarily taking the form of rigid or semi-rigid rings 26 provided along an inner side with a slotted or pierced web 28. Web 28 is made of an elastic silicone (silastic) material for facilitating the insertion of a shaft 30 of an ultrasonic instrument 32 and the utilization of the instrument in an ultrasonic tissue ablation or debridement procedure on wound WND. The elastic silicone material of web 28 enables the formation of a seal about instrument shaft 30. The silicone material is essentially impervious to the ultrasonic vibrations of the instrument 32. Port members 24 are thus effective to at least inhibit or reduce the escape of airborne particles from bag 10 while permitting penetration into the bag of surgical instrument shaft 30.

FIG. 3 depicts three alternative port members 34, 36, 38 that may be provided on bag 10 in place of or together with port members 24. Port member 34 comprises a ring 40 and a web 42 of self-sealing silastic material. Web 42 completely covers the respective opening 44 in bag 10, until the web is pierced during a surgical procedure to enable the passage of a surgical instrument.

Port member 36 comprises a ring 46 and a silastic web 48 including several flaps 50 separated from one another by slits 52 and connected at respective apices 54 by a button 56. multiple instruments may be inserted into bag 10 through slits 52.

Port member 38 includes a tubular component 58 connected to bag 10 via a ring 60. Tubular component 58 is optionally provided with a sealing web 62 and a drainage tube 64. In use, a lower rim (not separately designated) or ring 60 may be placed into contact with a patient's skin about a surgical site. A distal end portion of a surgical instrument is inserted into tubular component 58 and through web 62 to operate on tissues at the surgical site, at the base of tubular component 58. In the event that liquid accumulate in tubular component 58, owing to web 62 and the sealing engagement of ring 60 with the patient's skin, tube 64 is used to drain the collected liquid into bag 10.

A surgical fluids containment bag may take many different forms, depending on the location of the wounds to be treated. FIG. 5 shows a bag 66 in that has the shape of a pair of shorts with a waist opening 68 and two leg openings 70, each surrounded at its rim by a respective elastic band or drawstring 72, 74. Bag 66 is provided with multiple surgical access openings 76 defined by respective silastic port members 78. Bag 66 also has an opening 80 for liquid drainage, provided with a valve 82 and a connector 84.

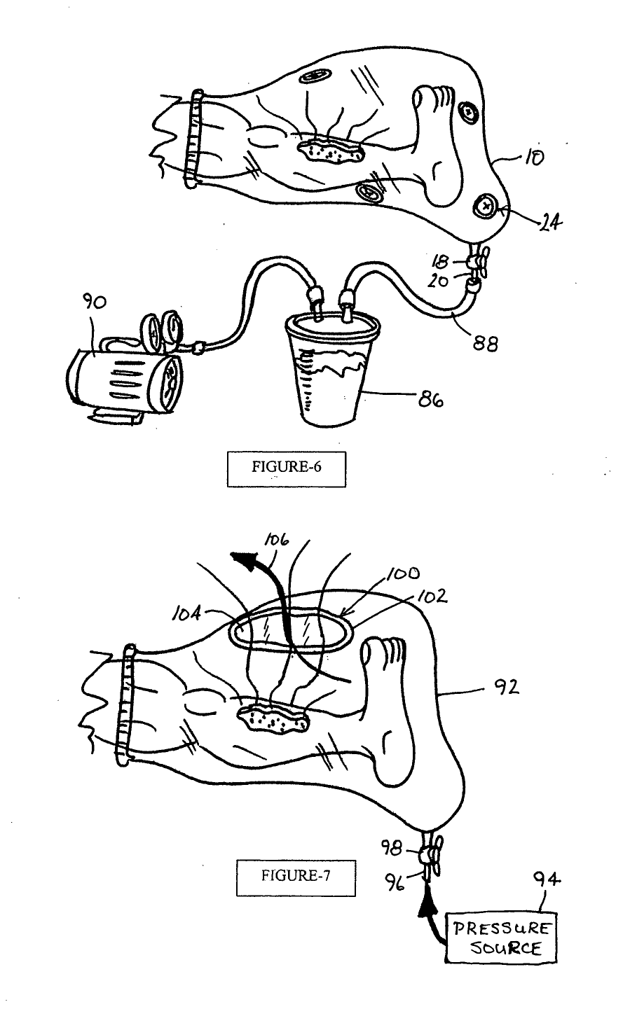

As illustrated in FIG. 6, valve 18 (or 82) may be connected to a liquid disposal container 86 via connector 20 (or 84) and a hose 88. A suction source or vacuum pump 90 is operatively connected to container 86 for extracting liquid via valve 18 (or 82) from a sump formed in bag 10 (or 66).

Typically, port members disclosed herein do not form air-tight seals at the respective surgical access openings in a fluids containment bag. Thus, when the bag is moved, air may be sucked in through the surgical access openings to enable the maintenance of a work space in the containment bag about the surgical site. These considerations apply to port members 24, 36, 38 and 78.

As illustrated in FIG. 7, it may be desirable to provide a positive pressure to a fluids containment bag 92. Accordingly, a source 94 of pressurized gas such as air or carbon dioxide may be coupled to bag 92 via a connector 96 and a valve 98. Bag 92 is provided a surgical access port 100 including a ring 102 and a silastic sealing element 104 into the form of one or more flaps or a perforated web member. Sealing element 104, while preventing or inhibiting the escape of airborne particles such as atomized liquid, may permit the leakage of some air, as indicated by an arrow 106.

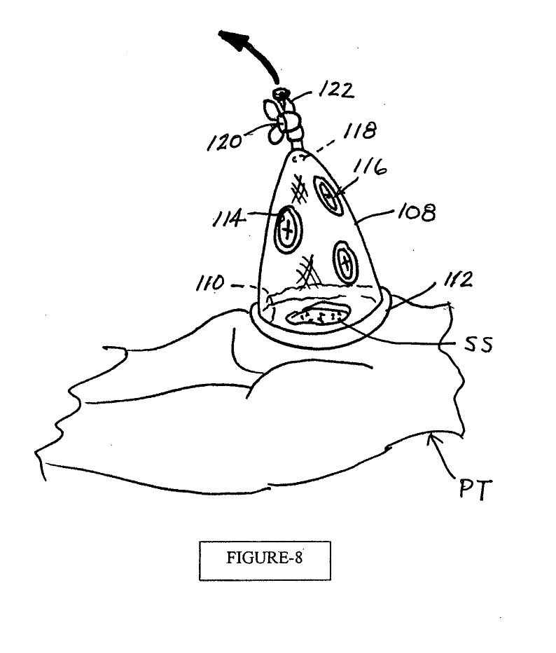

As depicted in FIG. 8, a fluids containment device includes a cup-shaped bag 108 provided with a first opening 110 surrounded at its rim (not designated) by a sealing element 112 exemplarily in the form of a ring provided with an adhesive layer (not illustrated) for forming a sealing engagement with the skin of a patient PT about a surgical site SS. Bag 108 is provided with a plurality of second openings or apertures 114 that are effectively closed to airborne particles by respective port members 116. Port members can take any suitable form, including any described hereinabove. Bag 108 includes a third opening 118 provided with a valve 120 and a connector 122 for enabling the removal of liquid waste material. A portion of bag 108 in a region about opening 118 may be disposed at a location lower than surgical site SS to enable the collection and evacuation of the liquid waste.

In a method utilizing a fluids containment system as described above, bag 10, 66, 108 is disposed about a portion of the patient's body having a target surgical site WND, SS. A rim of first opening 12, 68, 70, 110 is engaged with the patient's body to close off the opening. This closure may be effectuated in part by the nature of the band, drawstring, or adhesive seal 14, 72, 74, 112 about the opening 12, 68, 70, 110. A distal end portion of a surgical instrument 32 is inserted through at least an outer portion of port member 24, 34, 36, 38, 78, 100, 116 and thereafter the surgical instrument 32 is operated to effectuate a surgical procedure on tissues of the patient at the surgical site WND, SS. Valve 18, 82, 98, 120 is opened to drain liquid waste material from the bag 10, 66, 108.

In the case of port member 38 (FIG. 3), a rim of tubular element 58 may be placed into engagement with the patient's body about the surgical site WND or SS so that the distal end portion of surgical instrument shaft 30 is surrounded by the tubular member during the operating of surgical instrument 32. Fluid may be drained from tubular member 58 through drainage tube 64.

Instrument shaft 30 may be subjected to ultrasonic vibrations for purposes of ultrasonically ablating or debriding tissues at surgical site WND or SS. In that case, port members 24, 34, 36, 38, 78, 100, and 116 are made at least in part of an ultrasound impervious material such as an eleastic silicone material.

Where port member 24, 36, 38, 78, 100, or 116 includes at least one flap (e.g., flaps 50), the inserting of the distal end portion of the surgical instrument 32 includes moving the flap. The flap may be partially distorted in order to accommodate the passage of instrument shaft 30.

As depicted in FIG. 2, bag 10 (or any of the surgical containment bags disclosed herein) may be provided with one or more support members 124, 126 for maintaining the bag in an at least partially expanded configuration. Support members 124, 126 may be circular ribs made of a resilient material, as indicated in the drawing, the ribs being bonded to an inner or outer surface of the bag. Alternatively, support members 124, 126 may be parts of a larger interconnected cage or frame structure that is connected to the bag, for instance, by adhesive, heat or ultrasonic welding, etc. Preferably, the support structure is resiliently deformable for enabling transport of the bag in a compact collapsed configuration.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A surgical liquid containment system comprising: a bag made at least partially of optically transmitting material, said bag have at least one first opening defined by a rim sealingly engageable with a portion of a patient's body, said bag having at least one second opening provided with a respective port member effective to reduce escape of airborne particles from said bag while permitting penetration into said bag of a surgical instrument, said bag having at least one third opening for liquid drainage, further comprising a valve at said third opening for enabling drainage of liquid effluent from said bag via said third opening.

2. The containment system defined in claim 1 wherein said port member includes a tubular member.

3. The containment system defined in claim 2 wherein said bag is provided at said second opening with a drainage element.

4. The containment system defined in claim 3 wherein said drainage element is a tube.

5. The containment system defined in claim 2 wherein said port member further includes at least one flap.

6. The containment system defined in claim 1 wherein said port member is made at least in part of an ultrasound impervious material.

7. The containment system defined in claim 6 wherein said material is a silicone material.

8. The containment system defined in claim 1 wherein said port member includes at least one flap.

9. The containment system defined in claim 1 wherein said first opening is provided along said rim with a drawstring.

10. The containment system defined in claim 1, further comprising a connector at said third opening for coupling said valve to a container and optionally a suction source.

11. The containment system defined in claim 1, further comprising a source of gas pressure operably connectable to said bag for maintaining said bag in an at least partially expanded state during a surgical operation in which said bag surrounds a portion of the patient's body including a target surgical site.

12. The containment system defined in claim 1, further comprising at least one support member connected to said bag for maintaining said bag in an at least partially expanded configuration.

13. A surgical method comprising:
providing a surgical liquid containment system including a bag made at least partially of optically transmitting material, said bag have at least one first opening defined by a rim sealingly engageable with a portion of a patient's body, said bag having at least one second opening provided with a respective port member effective to reduce escape of airborne particles from said bag while permitting penetration into said bag of a surgical device, said bag having at least one third opening for liquid drainage, further comprising a valve at said third opening for enabling drainage of liquid effluent from said bag via said third opening;
disposing said bag about a portion of the patient's body having a target surgical site;
engaging said rim with the patient's body to close off said first opening;
after the disposing of said bag about said portion of the patient's body, inserting a distal end portion of a surgical instrument through at least an outer portion of said port member;
after the insertion of said distal end portion of said surgical instrument through said outer portion of said port member, operating said surgical instrument to effectuate a surgical procedure on tissues of the patient at said surgical site; and
opening said valve to drain liquid from said bag.

14. The method defined in claim 13 wherein said port member includes a tubular member, further comprising placing a rim of said tubular element into engagement with the patient's body about said surgical site said distal end portion of said surgical instrument being surrounded by said tubular member during the operating of the surgical instrument.

15. The method defined in claim 13 wherein said bag is provided at said second opening with a drainage element, further comprising draining fluid from said tubular member through said drainage element.

16. The method defined in claim 13 wherein said port member is made at least in part of an ultrasound impervious material, said surgical instrument taking the form of an ultrasound instrument, the operating of said ultrasound instrument including ultrasonically ablating selected tissues of the patient at said surgical site.

17. The method defined in claim 13 wherein said port member includes at least one flap, the inserting of said distal end portion of said surgical instrument including moving said flap.

18. The method defined in claim 13 wherein said first opening is provided along said rim with a drawstring, further comprising closing said drawstring about the patient's body.

19. The method defined in claim 13, further comprising forming a connection at said third opening coupling said valve to a container and optionally a suction source.

20. The method defined in claim 13, further comprising connecting a source of gas pressure to said bag and operating said source to deliver gas to said bag to maintain said bag in an at least partially expanded state during the operating of said surgical instrument.
